(19) United States
(12) Patent Application Publication
(10) Pub. No.: US 2016/0128834 A1
(43) Pub. Date: May 12, 2016

(54) SCAFFOLD IMPLANT SYSTEM

(71) Applicants: Meir YAKIR, Raanana (IL); Lotan GUREWITZ, Herev Le'et (IL)

(72) Inventors: Meir YAKIR, Raanana (IL); Lotan GUREWITZ, Herev Le'et (IL)

(21) Appl. No.: 14/899,668

(22) PCT Filed: Jun. 18, 2014
(86) PCT No.: PCT/IL14/50551

§ 371 Date: Dec. 18, 2015

Related U.S. Application Data
(60) Provisional application No. 61/836,933, filed on Jun. 19, 2013.
(60) Provisional application No. 61/856,893, filed on Jul. 22, 2013.
(60) Provisional application No. 61/895,146, filed on Oct. 24, 2013.

(51) Int. Cl. A61F 2/28; A61F 2/30; A61C 8/00; A61F 2/44

(52) U.S. Cl. CPC A61F 2/2803; A61F 2/28; A61F 2/44; A61F 2/3099; A61C 8/0078; A61C 8/0022; A61C 8/0019; A61F 2002/2835

(57) ABSTRACT

The present invention is directed to isolated scaffold implant system comprising a stable combination of an implant fixture and a bone block, wherein said stable combination is suitable in size and form to be implanted within a prepared site in a subject's bone. The scaffold implant system of the present invention is particularly suitable for use in dental implant techniques.

### FIELD OF THE INVENTION

The present invention is directed to an implant system that is suitable for dental and surgical use. More specifically, the present invention relates to a highly stable bone block-implant fixture combination that may be used in a variety of dental and orthopedic implant procedures.

### BACKGROUND OF THE INVENTION

The use of dental implants in order to replace missing teeth has increased greatly in recent years. There are, however, certain technical and clinical problems which limit or prevent the use of implants in some circumstances. In particular, the insertion of dental implants into portions of the mandible or maxilla in which there has been bone loss often requires multiple procedures in order to prepare a stable implantation site. Similarly, additional preparatory techniques may also be required in cases wherein anatomical or pathological features of the adjoining sinuses result in the need for procedures that augment the amount of bone in the maxilla—for example the sinus lift procedure.

There have been some attempts in the prior art to solve these problems by means of implanting allogenic bone blocks into the site requiring augmentation, and subsequently performing an osteotomy and implantation of a titanium implant within the drilled block (see for example: Nissan J. et al., 2011, Clinical Implant Dentistry and Related Research, 13: 279-285).

These methods, however, suffer from a number of serious drawbacks. Firstly, the patient is required to make multiple visits, often separated by several weeks or months, in order to undergo the various steps of the augmentation and implantation procedures. Such multiple treatment steps increase the cost, morbidity and inconvenience to the patient. Another—and perhaps even more serious—problem associated with these prior art solutions is the poor mechanical stability of the titanium implant when using particulate bone substitute material or within the bone blocks. This problem arises from the fact that said implant is inserted by the clinician into the bone block in situ, within the patient's mouth. Not only is access to the block restricted (since it has already been implanted within the patient's maxillary or mandibular bone), but the magnitude of mechanical force (e.g. torque forces, in the case of screw-threaded implants) that may be applied by the clinician is obviously very limited. An attempt to place the implant fixture in the bone block requires manipulation of the block by the dentist. This is time consuming and technique sensitive. Less than optimum stability of the implant fixture within the implanted bone block will obviously result in poor functioning of the dentition, as well as a significantly-reduced success rate for the implant.

Similarly, in the field of orthopedic implants—and in particular, spinal implants—the prior art systems and methods are limited in their application and effectiveness by problems associated with the need for replacement bone blocks to be manipulated at the operating site.

There is thus a clear need for an implant system that is particularly suitable for use in situations in which bone augmentation may be required, and which overcomes the above-described drawbacks of the prior art systems. The present invention provides a novel and inventive solution to these drawbacks, as will be explained in more detail hereinbelow.

### SUMMARY OF THE INVENTION

The present invention is primarily directed to an isolated scaffold implant system comprising a stable combination of an implant fixture and a bone block, wherein said stable combination is suitable in size and form to be implanted within a target site in a subject's bone. Generally, the target site is prepared by the clinician in order to be able to receive the scaffold implant system.

The term “subject” is used herein to refer to a mammalian subject, preferably a human subject.

The system disclosed herein is particular suitable for use in the fields of dental implants and orthopedic implants. In much of the disclosure that follows, the implant system of the present invention will be described in relation to dental applications. However, it is to be recognized that said implant system is not restricted in its use only to dental implantation procedures, but rather is suitable also for use in implantation procedures in other body sites and systems. In particular, the presently disclosed and claimed implant system is highly suitable for use in orthopedic implantation procedures, particularly in the field of spinal implants.

Thus, in one highly preferred embodiment, the present invention is directed to an isolated dental implant system comprising a stable combination of an implant fixture and a bone block, where said stable composition is suitable in size and form to be implanted within (or adjacent to) the alveolar ridge in a subject's maxilla or mandible. Said implant fixture may be either fully or partially embedded within said bone block. For the sake of convenience, this implant system—comprising both a fixture and a bone block in stable combination—will be referred to herein as a ‘scaffold implant’ or ‘scaffold implant system’.

The term “isolated”, as used herein, is to be understood to refer to the fact that the combination of bone block and implant fixture is manufactured as a single, integral unit that may be sterilized, packaged and distributed in that form. This term is applied to the presently-disclosed system in order to clearly distinguish it from other combinations of bone blocks and dental implant fixtures that are created within the tissues of a subject undergoing a dental implant procedure, as known from the prior art.

The term “stable combination”, as used herein, is to be understood to refer to the fact that integral implant fixture—bone block combination is mechanically stable, to the extent that it is able to withstand all normal physiological forces applied thereon following implantation into, for example, the subject's jaw bone.

Any suitable source of bone may be used in order to manufacture the bone block of the present invention, including (but not limited to) allograft, xenograft or alloplast bone material. An important feature of the present invention is that the bone block used to produce the scaffold implant may be created in any desired shape or form, such that the clinician may choose from a range of bone block shapes, as required by the specific clinical situation.

The implant fixture portion of the scaffold implant system of the present invention may be manufactured from any suitable biocompatible material. In a particularly preferred embodiment, the biocompatible material is a biocompatible metal, most preferably titanium, alloys or mixtures containing titanium, zirconia and combinations of titanium and zirconia.

The implant fixture portion of the scaffold implant system may have any suitable shape and form, as desired for the particular clinical situation. Non-limiting examples of such suitable shapes and forms include: threaded root, threadless root, blade shape, cylindrical shape, T-shape and wedge shape.

The implant may be connected to the bone block at the time of manufacture by various means and techniques. Non-limiting examples of such means and techniques include: mechanical friction (i.e. application of compressive forces along the long axis of the implant fixture), application of torque forces (e.g. in the case of threaded fixtures), bone screws, and the use of cements, glues and other adhesives. In other cases, rather than inserting the implant fixture into a solid bone block, the block may be created by the addition of liquid or particulate bone to the fixture within a mold.

In certain preferred embodiments of this aspect of the invention, the implant fixture may also comprise one or more extensions, preferably ring-shaped extensions (although other shapes are also included within the scope of the invention) and wherein each of said extensions comprises an aperture suitable for containing and passing a bone fixation screw.

In some preferred embodiments, the implant fixture and/or the bone block comprise one or more transverse channels or holes suitable in size and shape for containing a bone fixation screw. In one preferred embodiment, said channels or holes pass through the entire thickness of the bone block as well as through the entire thickness of the metallic implant fixture. Further details of these bone fixation elements will be provided hereinbelow.

In other preferred embodiments, the scaffold implant system may further comprise additional means for securing the scaffold implant to the subject's bone. Non-limiting examples of such additional securing means include covering screws and healing abutments.

A key feature of the presently-disclosed and claimed scaffold implant system is the fact that the insertion of the fixture into the bone block is performed in the manufacturing facility, rather than in situ within the patient's mouth before implantation (as with current systems), thereby permitting greater mechanical forces to be applied in order to ensure an implant fixture—bone block combination having a high level of mechanical stability, with no or negligible movement between said fixture and said bone block. In one preferred embodiment, the mechanical forces applied to the fixture during the insertion thereof into the bone block are torque forces. In one particularly preferred embodiment, the mechanical force applied is at least 30N. In another particularly preferred embodiment, the mechanical force applied is at least 40 N.

In another aspect, the present invention provides a method for implanting a dental scaffold implant system as disclosed herein within the maxilla or mandible of a subject in need of such treatment, wherein said method comprises the steps of:
a) Preparing a site within, or in proximity to, the subject's alveolar bone in order to receive said implant system;
b) Inserting said implant system within the prepared site; and
c) Securing said implant system within the subject's maxillary or mandibular bone at the prepared site.

The implant system may be secured within the subject's bone using any suitable technique well known in the art, including, but not limited to mechanical friction contact, fixation membrane, bone screws, tacks, biological glue or titanium pins. In one particularly preferred embodiment of the method, the step of securing the scaffold implant within the subject's bone comprises inserting and tightening a bone screw through a pre-existing hole or channel that passes through the scaffold implant bone block and the implant fixture, and enters the subject's bone. The term “pre-existing” refers to the fact that the hole or channel was drilled through bone block and titanium implant fixture during the manufacture of the scaffold implant, and not as part of the clinical implantation procedure.

Following implantation of the scaffold implant system of the present invention, a dental restoration (such as an artificial crown, bridge or denture) may be attached to said scaffold system, preferably by means of inserting a portion of said restoration into the central bore of the implant fixture and securing same therein.

It should be appreciated, as explained hereinabove, that the scaffold implant system of the present invention is also suitable for use in the field of orthopedic implants, in particular, spinal implants.

### BRIEF DESCRIPTION OF THE DRAWINGS

FIG. 1 depicts, in top view, a scaffold implant of the present invention incorporating a screw-thread implant embedded within a bone block.

FIG. 2 shows in vertical section view the same embodiment that was shown in FIG. 1.

FIG. 3 pictorially illustrates examples of scaffold implants, each having different implant fixture geometries, following their implantation into a subject's mandible.

FIG. 4 provides a cut-away view of a scaffold implant having a fully embedded implant fixture.

FIG. 5 provides a longitudinal section view of a partially-embedded wedge shaped implant fixture following its insertion into a bone block.

FIG. 6 shows a transverse section view of the embodiment depicted in FIG. 5.

FIG. 7 depicts, in longitudinal section, an alternative embodiment of a partially-embedded implant fixture, characterized by having a T-shaped extension.

FIG. 8 provides a top view of the embodiment shown in FIG. 7.

FIG. 9 shows an example of a fully-embedded scaffold implant fitted with an extension ring and two bone screw apertures.

FIG. 10 illustrates a further embodiment of the present invention in which a bone screw is used to secure the scaffold implant into the patient's maxillary or mandibular bone.

FIG. 11 depicts another embodiment of a scaffold implant of the present invention, comprising both a long bone screw for fixation into the patient's jaw bone and a shorter bone screw for providing further stabilization of the metallic implant fixture within the bone block.

FIG. 12 provides a longitudinal section of a further embodiment of the scaffold implant, in which the implant fixture comprises a horizontally-disposed extension ring.

FIG. 13 provides a top view of the embodiment illustrated in FIG. 12.

FIG. 14 shows, in both plan view and side view, a covering screw for use in conjunction with the scaffold implant of the present invention.

FIG. 15 shows an example of a healing abutment that may be used in order to provide further stabilization of the scaffold implant of the present invention within the patient's mandible or maxilla.

FIG. 16 illustrates a further embodiment of a healing abutment for use with the scaffold implant of the present invention, wherein the abutment comprises two separate parts: an upper plate and a connecting screw.

FIG. 17 depicts a scaffold implant of the present invention in which a single structure comprising a titanium mesh and two implant fixtures connected thereto is embedded within a bone block.

### DETAILED DESCRIPTION OF THE PREFERRED EMBODIMENTS

As disclosed hereinabove, the dental implant system of the present invention comprises a bone block into which an implant fixture has been inserted, either partially or fully, during the manufacturing process in such a way that each of the two components of said system (i.e. the fixture and the bone block) is essentially immovable in relation to the other component.

The implant fixture body of the present invention may be made of any suitable biocompatible material having the required physical properties, as well known in this field. Typically, however, the implant fixture body will be constructed from titanium or zirconia, or combinations thereof.

The implant fixture body can vary in shape, form, width and length. In one preferred embodiment, it can be in the form of a root form screw type implant of regular size, having, for example, a diameter between about 3.0 mm and 6.0 mm, and a length in the range of about 5.0 mm to 15.0 mm.

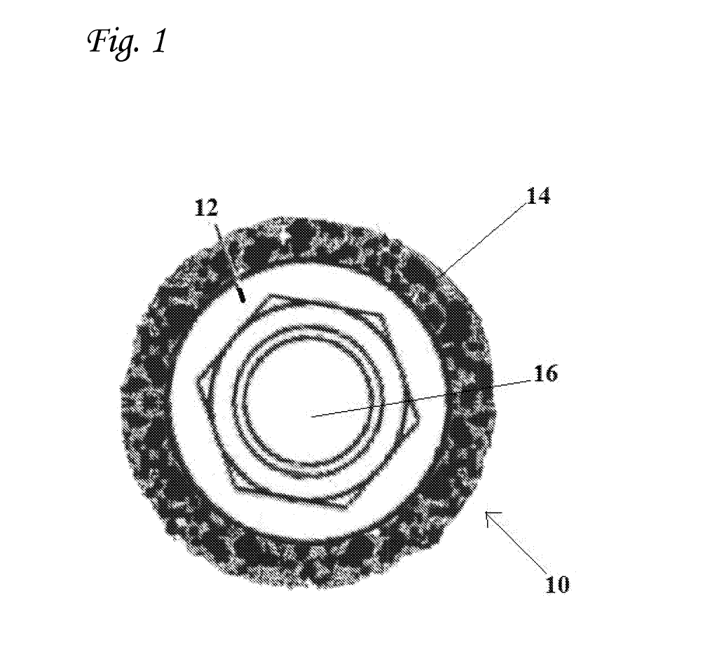

An example of a scaffold implant that incorporates a screw-thread type of implant fixture is shown in top view in FIG. 1. It may be seen that in this particular type of scaffold implant 10, both the implant fixture 12 and the bone block 14, in which it is inserted have a circular cross section. It is to be recognized, however, that one of the key advantages of the present invention is that the manufacture of the fixture-bone block assembly in the factory (rather than at the chair-side) permits a variety of different cross-sectional geometries (with regard to both the fixture and the bone block) to be created. The central portion 16 of the fixture is hollow and is adapted to receive a crown or other dental prosthesis.

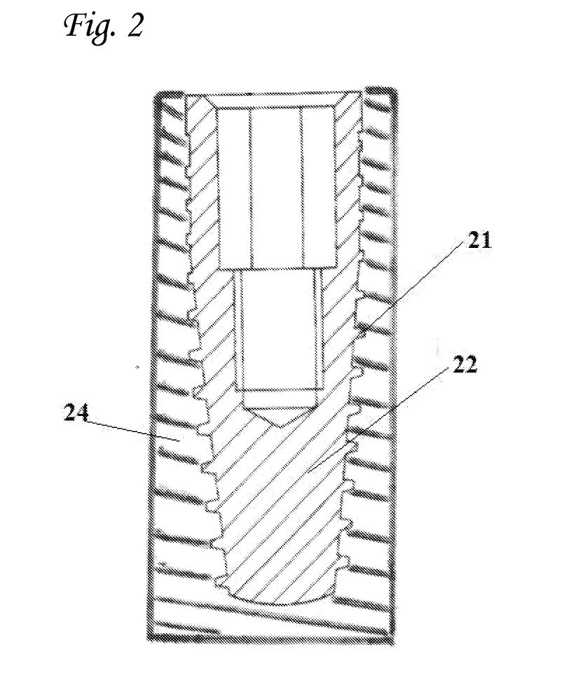

The same implant fixture depicted in FIG. 1 is also shown in vertical section view in FIG. 2. It may be seen in this figure that the screw thread 21 of the implant fixture 22 has been firmly secured into bone block 24.

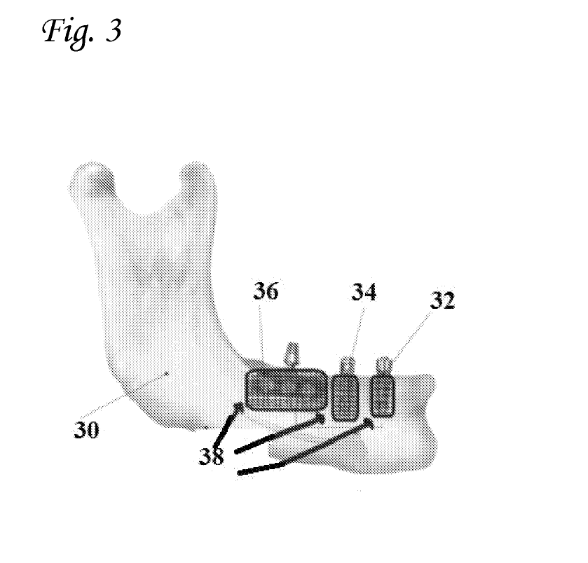

In other preferred embodiments, the implant fixture body may have, for example, a threadless root form, a blade shape, T-shape or a wedge shape, in accordance with the needs of the particular clinical situation. Three different examples of such fixtures following insertion of the scaffold implant into the mandible 30 are shown in FIG. 3: screw-thread fixture 32, cylindrical fixture 34 and blade fixture 36. In each case, the bone block portion of the scaffold implant is represented by a rectangle 38 which indicates the size and orientation of the block that may be used in conjunction with each of the representative fixtures shown in this figure. It is to be emphasized that these are only examples of some of the possible shapes and forms of implant fixture body, and that other shapes and forms thereof are also included within the scope of the present invention.

In order to manufacture the implant system of the present invention, the implant fixture may be custom-produced from medical-grade titanium, zirconia or a similar metal or alloy using any of the production techniques well known in this technical field, including, but not limited to, machining, casting, 3D printing, computer numeric control (CNC) milling, and the like.

Thus, in one preferred embodiment, the implant fixture is produced using an additive manufacturing (AM) technique, such as 3D printing, which permits the creation of a three-dimensional solid object of virtually any shape from a digital model. 3D printing is achieved using an additive process, where successive layers of material are laid down in different shapes.

In another embodiment, the manufacturing technique used is Numerical control (NC), in which the machine tools are operated by precisely programmed commands encoded in a storage medium. Most NC in current use relies on computer numerical control (CNC), in which computers play an integral part of the control. A distinct advantage of using CNC in the manufacture of the presently-claimed scaffold implant system is that the series of steps needed to produce said system is highly automated, thereby enabling a very close match between the final product and the original computer aided design (CAD).

Alternatively, the implant fixture used in the manufacture of the presently-disclosed system may be a commercially-available dental implant fixture, as well known in the art. Non-limitative examples of such implant fixtures include the Screw Vent made by Zimmer Dental Inc., (USA), Nobel Active made by Nobel Biocare AB, (Switzerland), Seven made by MIS Implant Technologies, (Israel) and ICE produced by Alpha Bio Tec. (Israel).

The bone block is produced using bone of any suitable type and origin, including—but not limited to—xenografts, allografts, coral, and alloplasts such as TCP and Hydroxyapatite. The block itself is preferably prepared using the same techniques (e.g. AM, NC) used to manufacture the implant fixture. The use of these techniques permits the bone block to be formed into any desired shape.

The two components of the combined product—that is, the bone block and the implant fixture—may, in one preferred embodiment, be assembled by applying controlled pressure to insert the fixture portion into a matching space in the bone block, using friction in order to achieve stability. In one preferred embodiment, the implant fixture is screwed into place in the bone block with controlled force. Preferably, this force has a value of at least 30N.

In a further preferred embodiment, the scaffold implant device is manufactured by using bone material (xenografts, allografts, coral, and/or alloplasts) that is ground into particles. This particulate bone is then poured over the metal implant fixture covering all or parts of the fixture and then solidifying it using a binder. It also can be in form of injectable and moldable paste that hardens to a porous body over the implant fixture. A commercially-available example of such particulate bone is Easy-Graft (Degradable Solutions AG, Switzerland).

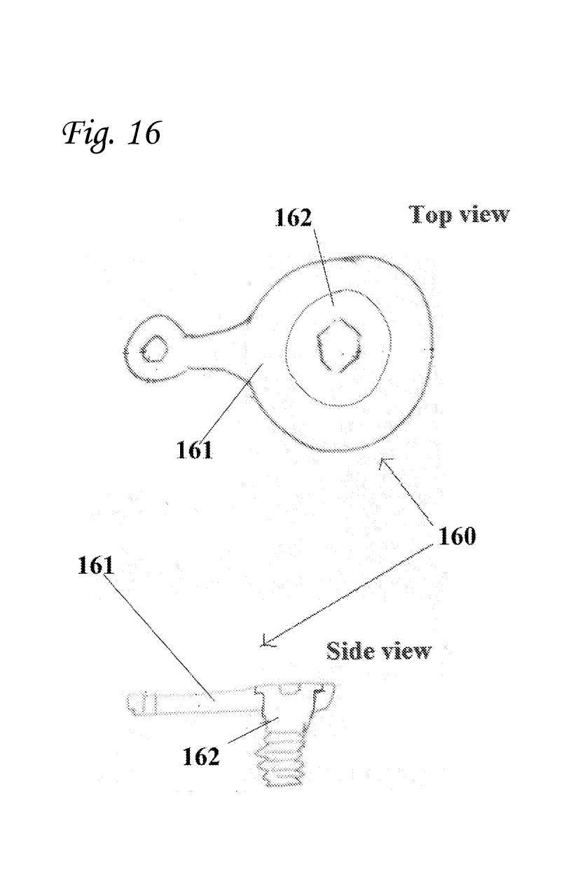

In one version of this method of preparing the scaffold implant, 3D printing techniques are used in order to create an implant comprising bone, a titanium mesh and a titanium fixture body that is suitable for receiving a dental restoration. In this way, the scaffold implant may be manufactured as a single unit, using a single manufacturing technique. An example of this type of manufacturing approach is described in Example 3, hereinbelow, with reference to FIG. 16.

In another version of this preferred embodiment, the titanium fixture is composed of mesh and, together with the attached implant restorative connection, may be manufactured, in one step, using 3D printing. This titanium mesh fixture is then placed in a mold, following which liquid or particulate bone is poured over said titanium mesh fixture within said mold, thereby creating a scaffold implant comprising a titanium mesh fixture embedded within a bone matrix.

The assembly and subsequent packaging of the scaffold implant is preferably performed in a clean room, as is well known to the skilled artisan in this field.

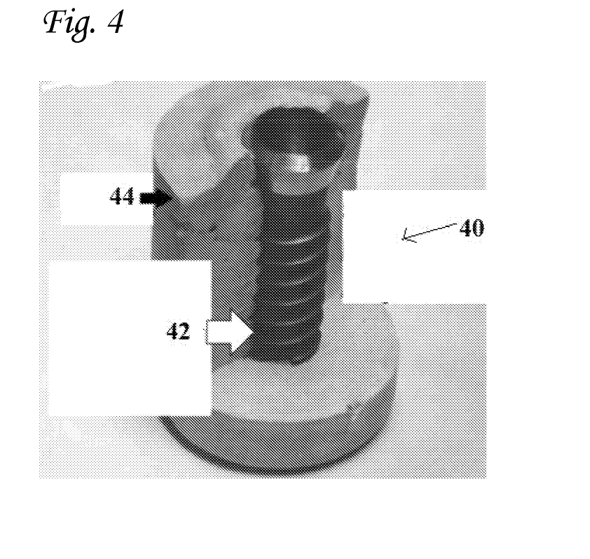

The bone block portion of the scaffold implant system may be prepared from allograft, xenograft or alloplast bone material, and may be prepared in various different shapes, including (but not limited to) round, square, rectangular, and so on. The scaffold implant with a fully embedded implant fixture will have a bone block of about 1.0 mm-15.0 mm thickness all around the implant fixture. One example of a fully-embedded fixture 40 is shown in FIG. 4, which presents a cut-away view of a screw-thread device 42, inserted within circular bone block 44. Approximately half of the bone block has been removed in order to illustrate that the fixture is completely embedded, along its entire length within the center of the bone block.

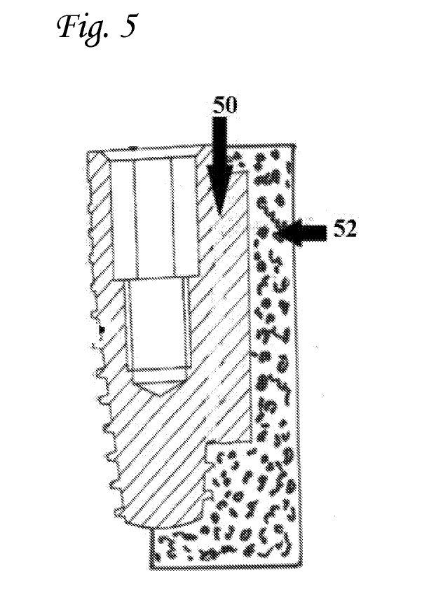

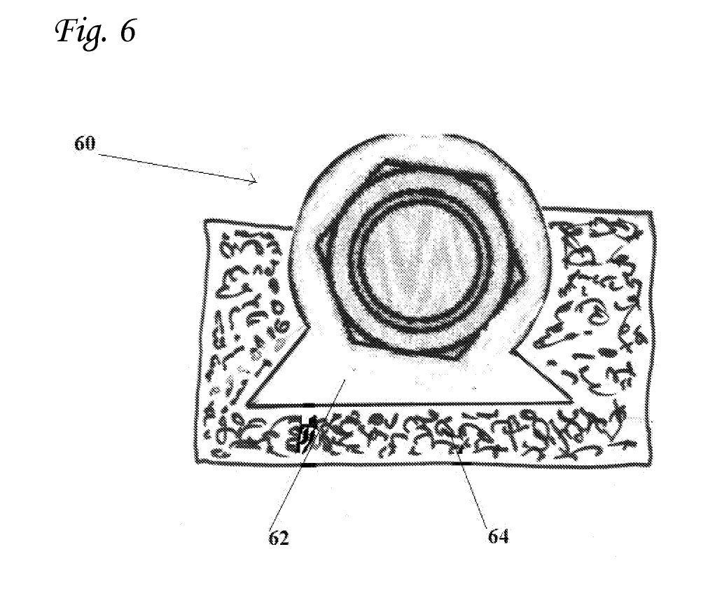

In scaffold implants with a partially embedded implant fixture, the exposed portion of the implant fixture will be in direct contact with the patient's bone. Two different examples of scaffold implants having partially embedded fixtures are shown in FIGS. 5 to 8. Thus, FIG. 5 provides a longitudinal section view of a wedge-shaped fixture 50 that is partially embedded within bone block 52. The same embodiment is also shown in FIG. 6, but this time in transverse section, looking from above. It may readily be seen from this figure that the wedge-shaped portion 62 of the metallic implant fixture 60 is firmly held in place within a matching undercut portion of bone block 64.

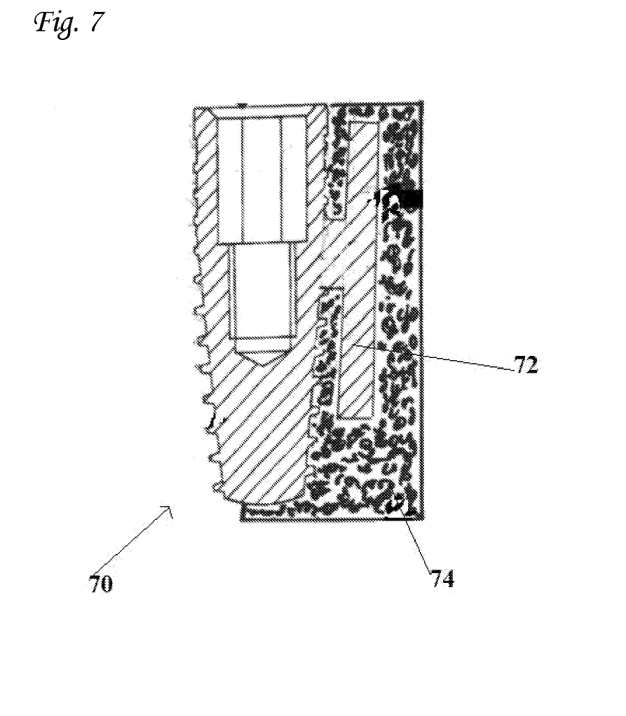

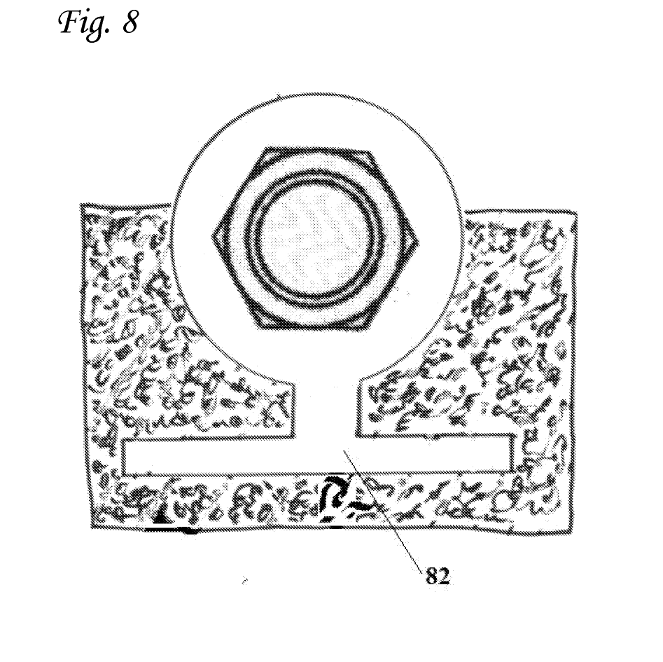

An alternative embodiment of a partially-embedded implant fixture is depicted in longitudinal section in FIG. 7, which shows a T-shaped portion 72 of implant fixture 70 embedded with a rectangular recess cut in bone block 74. A top view of a transverse section of this embodiment is seen in FIG. 8, and illustrates the fact that T-shaped portion 82 extends both laterally—as shown in this figure—and longitudinally (as shown in FIG. 7).

Generally (i.e. regardless of whether it is fully- or partially-embedded), the implant fixture head is free of bone to enable the receiving of a prosthetic restoration therein.

The scaffold implant system—that is the combination of implant fixture and bone block—is assembled by the manufacturer in commercial conditions, rather than in a clinical setting. The shape and size of the bone block is accurately created and controlled, and the implant fixture is inserted into its intended position within the bone block, such that the stability of the fixture-bone block combination is maximized.

In the case of screw-threaded implants, a predetermined torque is used in order to achieve stable insertion of the implant fixture within the bone block. One significant advantage of the present invention in relation to prior art products and methods is that this insertion process is performed in a controlled environment, wherein precisely the correct amount of torque force may be applied in order to achieve the desired result. This is in marked contrast to the prior art techniques, in which the implant fixture is inserted and stabilized within the bone block after the latter has been implanted within the patient's mouth. Such prior art methods clearly pose significant problems with regard to the insertion of screw-threaded implants (and also of other forms of implant) into the bone block, including, for example the dislodgement of the bone block from its augmented position when forces are applied thereon in order to stabilize the implant fixture. The present invention provides a novel and inventive solution to these problems.

In some embodiments of the present invention, the implant fixture may be secured to the bone block with a screw.

The scaffold implant (i.e. the combination of the implant fixture and bone block) is implanted by the clinician into the intended working site as a single, integral unit.

At the time of implantation the intended site in the mandible or maxilla is prepared for receiving the scaffold implant according to the shape and size of the bone block, the implant fixture (for the partially embedded type), and the shape of the alveolar ridge. The scaffold implant is placed in contact with the patient's bone surface of the osteotomy site and secured in place by means of any suitable method, as well known in the art, including (but not limited to) mechanical contact, membrane, bone screws, tacks, biological glue or titanium pins.

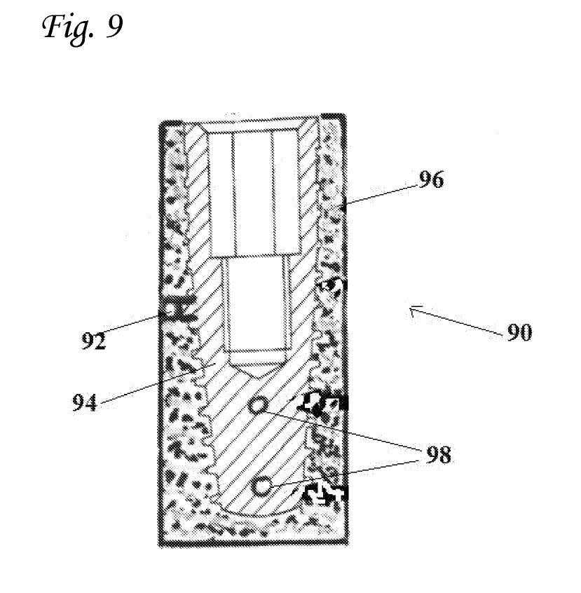

In some preferred embodiments, the bone block may be further stabilized with either a ring-shaped extension from the implant fixture body or a channel within the implant fixture body, both of which structures provide an aperture for receiving a bone screw. Thus, in one embodiment, the bone screw is placed through the bone block, into the implant fixture extension ring or channel and then into the patient's bone. A predetermined channel is made in the bone block to receive the bone screw and to guide it through the implant fixture channel or extension ring. FIG. 9 shows an example of a fully-embedded scaffold implant 90 of the present invention fitted with an extension ring 92, passing from the titanium implant fixture 94 into bone block 96. The figure also shows two bone screw apertures 98, each of which represents the lateral openings of a bone screw channel.

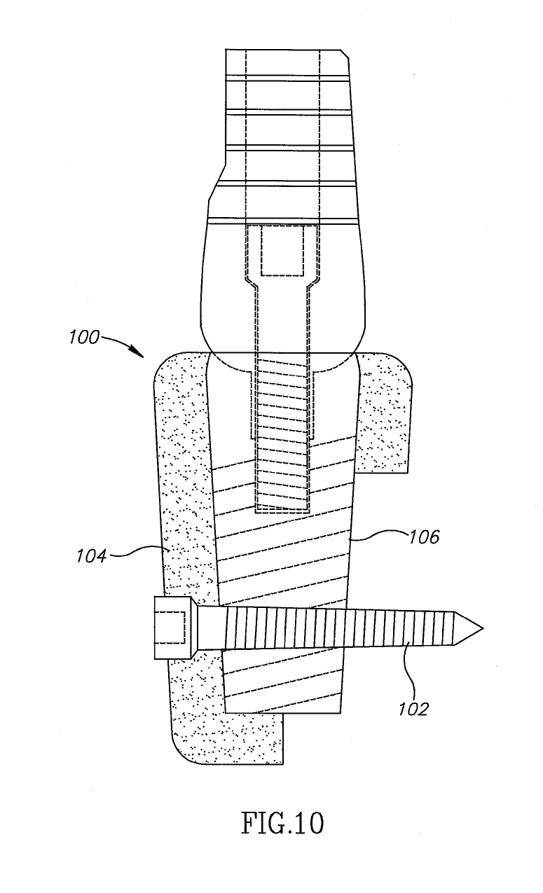

A sagittal section of a similar embodiment is illustrated in FIG. 10. In the scaffold implant 100 shown in this figure, a long bone screw is seen passing sequentially (from the buccal to lingual side) through the thickness of bone block 104, a channel in screw-thread implant fixture 106. The remaining distal portion of the bone screw thread (i.e. the length of the bone screw thread that emerges from the second side of the implant fixture is intended to be used to secure the entire scaffold implant 100 into the patient's mandibular or maxillary bone.

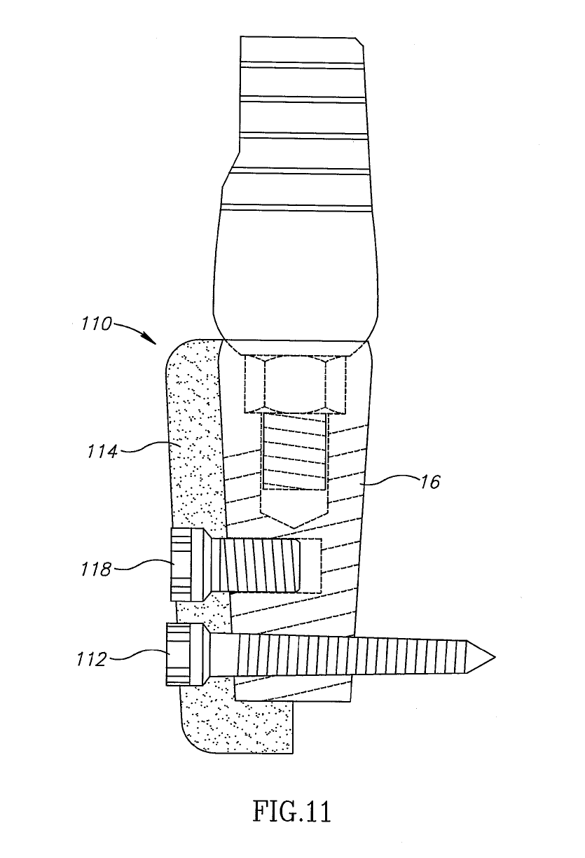

A further embodiment of a screw-affixed device is shown in FIG. 11. In this embodiment (as in the device shown in FIG. 10), a long bone screw 112 is used to assist in securing scaffold implant 110 into the patient's jaw bone. In addition, a second, shorter bone screw 118 is used, at the time of manufacture of the scaffold implant, in order to provide additional stabilization of the implant fixture 116 within bone block 114.

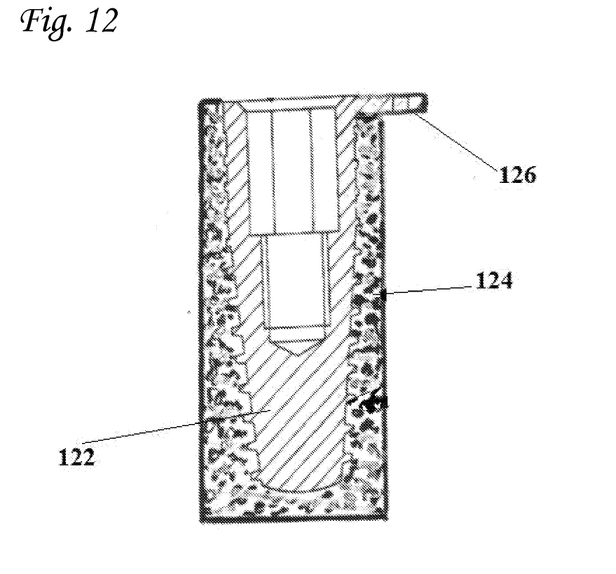

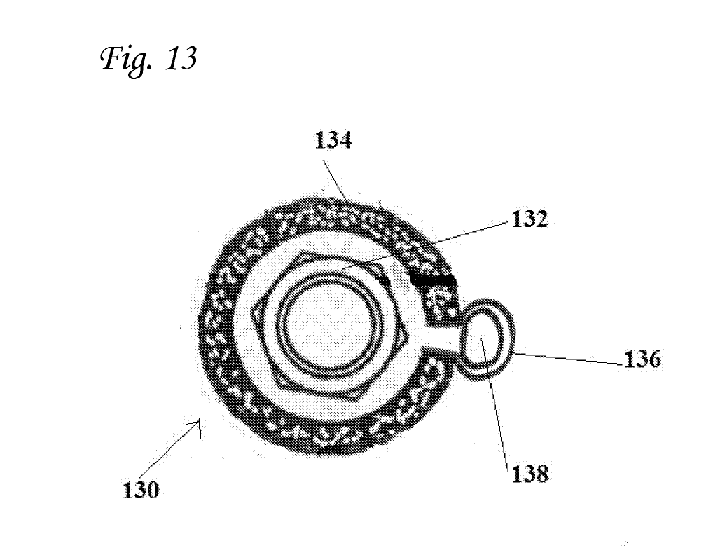

In another preferred embodiment of the present invention, as shown in longitudinal section in FIG. 12, the metallic implant fixture 122 further comprises an extension ring 126 continuous with the proximal (upper) portion of one side of the outer wall of said fixture. Said extension ring, passes horizontally through a groove or channel cut in bone block 124. As seen in the top view of this embodiment of the scaffold implant 130 (shown in FIG. 13), the extension ring 136 is perforated by an aperture 138 such that a bone screw passing vertically through said aperture may be secured into the patient's maxillary or mandibular bone, thereby providing further stabilization of the entire scaffold implant (i.e. the combination of implant fixture 132 and bone block 134) following implantation.

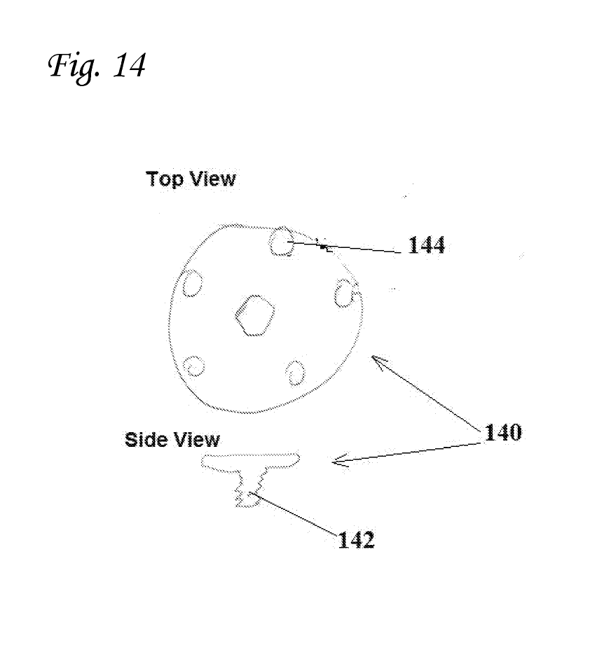

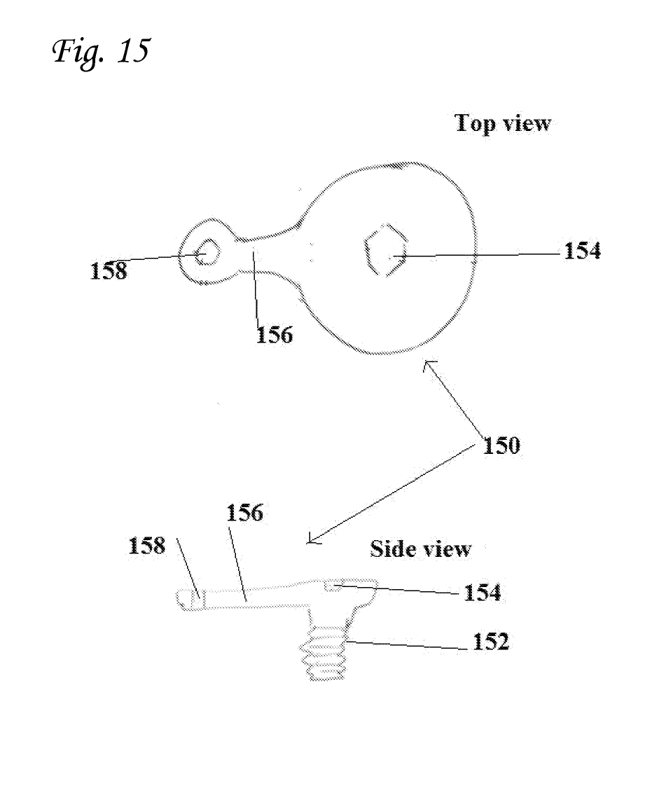

In an alternative embodiment, the scaffold implant may be secured to the patient's bone with bone screw that is placed through an aperture in a covering screw or healing abutment connected to the implant fixture, as exemplified in FIGS. 14 and 15, respectively. Thus, FIG. 14 provides both a plan view and side view of a typical covering screw 140, which comprises a screw thread 142 used to retain said covering screw within the central bore of the implant fixture. In addition, covering screw 140 is perforated by a plurality of screw holes 144, through which bone screws (not shown) are inserted, said bone screws then being screwed into the patient's jaw bone in order to provide further stabilization of the scaffold implant.

In a further embodiment, as shown in FIG. 15, a healing abutment is provided in order to provide further stabilization of the scaffold implant within the patient's jawbone. Thus, this figure depicts (in both plan and side views) healing abutment 150 having a vertical screw thread 152, used to retain said abutment within the central bore of the implant fixture. The upper surface of the abutment is perforated by a tooling aperture 154, which is used to facilitate the use of a screwdriver or similar tool inserted therein in order to insert or remove said abutment. A lateral extension 156 that is continuous with the main body of abutment 150 is perforated at its lateral extremity by aperture 158, through which bone screws may be inserted in order to anchor the abutment (and hence the entire scaffold implant) into the patients mandible or maxilla.

In another preferred embodiment, the invention may comprise a healing abutment very similar in structure to that described hereinabove (and depicted in FIG. 15). However, in the case of this embodiment, as illustrated in FIG. 16, the healing abutment 160 comprises two separate portions—an upper plate 161 and a connecting screw 162.

In some preferred embodiments, a barrier membrane may be secured to the scaffold implant, and as such will form part of the implant system of the present invention. The strategy of isolating the bone block with a mat-like material (resorbable or non-resorbable) that will function as a physical barrier to avoid gingival cell invasion led to the development of GTR/GBR membranes. Membranes for GTR/GBR procedures currently on the market are high-density polytetrafluoroethylene, PTFE (e.g., Cytoplast® TXT-200, Osteogenics Biomedical, Lubbock, Tex., USA). Titanium-reinforced high-density polytetrafluoroethylene (e.g., Cytoplast® Ti-250, Osteogenics Biomedical, Lubbock, Tex., USA). The majority of synthetic polymer resorbable membranes for periodontal regeneration on the market are either based on polyesters (e.g., poly(glycolic acid) (PGA), poly(lactic acid) (PLA), poly(-caprolactone) (PCL), and their copolymers), or tissue-derived collagens. Non-limitative examples include Alloderm® and LifeCell (Branchburg, N.J., USA), bovine Achilles tendon (Cytoplast® Collagen, USA) and porcine skin (Bio-Gide®, Osteohealth, Shirley, N.Y., USA).

The main disadvantage encountered in the prior art use of such membranes is the time consuming manipulation required by the dentist following bone augmentation, since said membrane needs to be cut to a specific size and then placed and secured in a specific position.

The scaffold implant device of the present invention may itself comprise a barrier membrane attached to part or all of the external surface of the scaffold implant (i.e. attached to the bone block and/or implant fixture). In this way, the presently-disclosed device incorporating a barrier membrane at the manufacturing stage is able to solve the above-described problem, since said membrane can be trimmed to size and connected to the bone block at the manufacturing stage or when assembled and packaged in the clean room.

The above-described membrane may be folded and cemented, bonded or by any other means connected to the Scaffold Implant such as with tacks, screws or pins. It can be unfolded, after securing the Scaffold Implant in its place within the jaw and also used to cover the Scaffold Implant. In such circumstances, the membrane may be trimmed to size in advance in order to fit the size of the Scaffold Implant.

In another preferred embodiment a liquid barrier membrane such as Atrisorb® FreeFlow™ manufactured by Tolmar Inc. (USA), may be used, wherein it is applied onto the bone block of the scaffold implant prior to packaging via dipping, painting, or spraying.

In further preferred embodiments, additional bone graft material can be added, if needed, in order to close gaps between the scaffold implant and the jaw bone.

The implant fixture may or may not be restored at the time of implantation. If not restored at the time of placement it can be restored approximately three to six months later, over the course of which time, the scaffold implant system will have been able to undergo satisfactory osseointegration.

Further advantages of the dental implant system of the present invention are summarized hereinbelow:
1) Alveolar bone loss often limits the ability to successfully place dental implants due to the absence of sufficient bone volume in the mandible or maxilla. In many cases, this deficiency is treated by bone augmentation procedures, often as a separate, additional surgical procedure prior to implant placement. The scaffold implant system of the present invention eliminates the need for a secondary surgical procedure, thereby saving operating room time and costs, as well as reducing pain and morbidity, and shortening the rehabilitation time for the patient.
2) The stability of the implant fixture within the bone block is always secured at the manufacturing stage. It therefore does not depend upon the amount or type of bone at the osteotomy site, or upon the clinician's own skills.
3) Micro-movement of the implant fixture immediately after implantation, which can often cause failure of prior art implant systems, has limited effect on the integration of the scaffold implant of the present invention, because said micro-movement will have no effect or only a negligible effect on the integration of the bone block in the patient's alveolar bone.
4) Unlike prior art systems, the presently disclosed implant system permits the placement of the scaffold implant outside the boundary of the patient alveolar ridge. Thus, for example, it may be placed adjacent to the buccal/lingual plate of said ridge with or without additional bone graft, provided that it is secured in place and that proper blood supply is maintained.
5) Increased vertical ridge height may be achieved by placing the scaffold implant above the crestal boundary with or without additional bone graft.
6) Using the presently-disclosed implant system, a sinus lift procedure can be performed without dependency on the Schneiderian membrane, and without the need for a lateral window approach. The scaffold implant can be inserted into the maxillary sinus via crestal ridge osteotomy with tearing of the Schneiderian membrane. Provided that the implanted scaffold implant has been secured in place, it will have the necessary bone environment to allow for proper integration.
7) In the case of buccal wall fracture during extraction of a tooth (e.g. prior to implant placement), the scaffold implant can be placed immediately, thereby eliminating the need for two separate procedures: bone graft placement and implant fixture placement.
8) The implant procedure using the presently-disclosed scaffold implant takes significantly less time to complete than using current methods, since the steps of creating a socket within the bone block, inserting the implant fixture within the bone block and tightening and stabilizing said fixture therein are all performed at the manufacturing site, rather than at the chair side and/or within the confines of the patient's mouth.

The presently-disclosed invention will now be described in more detail in the following working examples. It should be appreciated that these examples provide details of specific embodiments, and do not limit the scope of the claimed invention in any way.

### EXAMPLE 1

### Preparation of a Scaffold Implant System using a Bovine Bone Block

One embodiment of the scaffold implant device of the present invention may be prepared using a bovine bone block. A suitably-sized bone piece is obtained and then processed and decontaminated in the usual manner rendering it suitable condition for augmentation in the human body. In the present example, the bovine bone block is obtained from a commercial supplier (for example Bio-Oss by Geistlich Germany or SmartBone by IBI, Switzerland).

The bone block is cut and trimmed into a specific shape using CNC technology. The external shape of the block is cylindrical having a diameter of 7.5 mm and a length of 11.5 mm in length.

A longitudinal hole, having a diameter of 4.2 mm and a length of 10 mm is drilled in the middle of the cylinder from the top, downwards.

A longitudinal portion of the block is removed from its base. The length of the removed portion is 8.5 mm. The 3.0 mm of the upper part of the bone block are cylindrical and the lower 8.5 mm are half cylindrical.

A horizontal hole having a diameter of 1.5 mm is made 3.0 mm from the lower end of the bone block and in its middle. The hole pierces the entire thickness of the bone block.

The other part of the device, the implant fixture, is made from type V titanium alloy. CNC technology is used in order to create a cylindrical screw-type of fixture having a diameter of 4.2 mm and a length of 10 mm. The upper part of the screw contains an internal hexagon connection for the future dental restoration. This connection is similar to that used in prior art fixtures (such as Seven by MIS, Israel or Screw Vent by Zimmer Dental, USA).

A horizontal hole having an internal diameter of 1.5 mm is drilled at the lower end of the implant fixture, piercing the entire thickness of the implant fixture.

The implant fixture is inserted into the bone block from the top face of the block and along its length, being inserted into the 4.2 mm diameter hole made in the block. The implant fixture is then torqued into place with a 45 Ncm force. At that position the 1.5 mm diameter horizontal hole in the bone block corresponds to, and is continuous with, the 1.5 mm horizontal hole in the implant fixture.

Following packaging and sterilization the device is ready for use.

### EXAMPLE 2

### Implantation of a Bovine Bone Block Scaffold Implant System

The dentist prepares an osteotomy site by exposing the alveolar bone. Using a dental drill with a diameter of 4.2 mm and length of 7.0 mm a longitudinal concavity having a depth of 2.1 mm and a length of 7.0 mm is cut into the bone. The concavity begins at the marginal ridge and continues toward the basal bone.

A scaffold implant device (prepared as described in Example 1, hereinabove) is inserted in the concavity by placing the exposed dental fixture portion in tight contact with the alveolar ridge bone. The 3.0 mm length of the upper part of the device is positioned on top of the alveolar ridge.

A bone screw (Surgidoc, United Kingdom) is used to secure the device to the alveolar ridge. It is placed through the horizontal hall in the bone block continuing through the horizontal hole in the implant fixture and into the alveolar ridge to a depth of about 5.0 mm. The bone screw is then torqued into its place with lag screw technique.

### EXAMPLE 3

### Scaffold Implant Mesh Device

The Scaffold implant mesh device is constructed from a titanium fixture and bone block.

The titanium fixture sprawls over the entire Scaffold implant space in a 3D mesh like structure (e.g. honeycomb shape). The titanium mesh can include different types of restorative connections (e.g. internal hexagon, external hexagon connection).

The titanium restorative connections will be positioned at the flat upper portion of the implant, ready to accept the prosthodontic restoration.

The titanium mesh is manufactured using an NC technique as a single unit that includes a mesh titanium fixture with restorative connections.

The Scaffold implant mesh device is surrounded by 1.5 mm depth of bone material forming a floor and walls around the mesh fixture and open at the top (e.g. forming an envelope-like structure). The bone material used may be of any suitable source, such as xenograft, allograft or alloplast, and is formed into its final shape using CAD/CAM and CNC equipment.

The inner spaces of the bone structure (i.e. in which the titanium mesh is embedded) are then filled with a commercially-available filler bone substance (particulate bone graft or ceramic filler), by means of pouring said filler into the space covering the titanium mesh and then allowed to solidify.

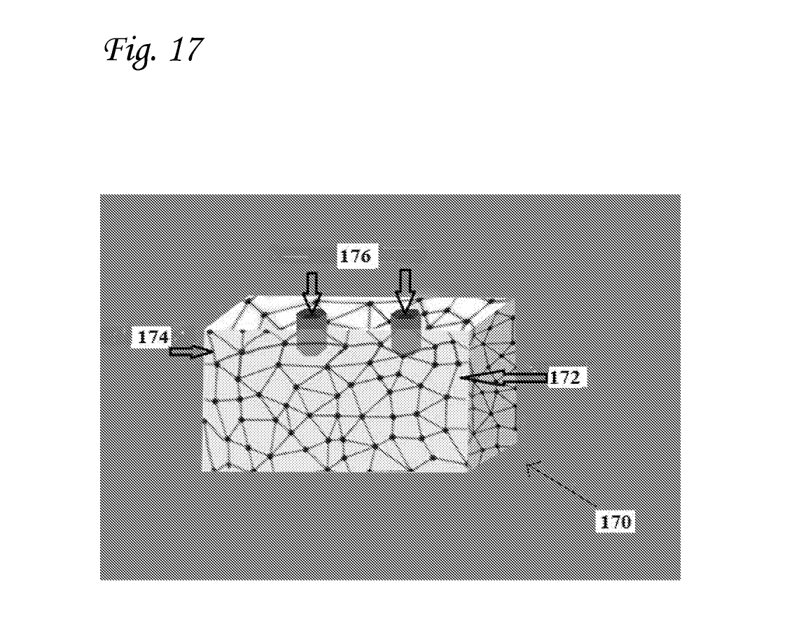

In the present example, illustrated in FIG. 17, the final scaffold implant 170, produced as described herein, comprises a cubic bone block 172, in which is embedded a titanium fixture comprising (as a single unit) a titanium mesh 174 and, in its upper portion, two implant fixture platforms 176 each comprising restorative connections, in order to enable the connection of suitable dental restorations to the scaffold implant, following its implantation.

1. An isolated scaffold implant system comprising a stable combination of an implant fixture and a bone block, wherein said stable combination is suitable in size and form to be implanted within a prepared site in a subject's bone.

2. The scaffold implant system of claim 1, wherein the said system is suitable in size and form to be implanted within a subject's maxilla or mandible.

3. The system of claim 1, wherein the bone block is obtained from allograft, xenograft or alloplast bone material.

4. The system of claim 1, wherein the implant fixture is fully embedded within the bone block.

5. The system of claim 1, wherein the implant fixture is partially embedded with the bone block.

6. The system of claim 1, wherein the implant fixture is connected to the bone block by means selected from the group consisting of friction, bone screw, cement, glue and addition of liquid or particulate bone to the fixture.

7. The system of claim 1, wherein the implant fixture is manufactured from a biocompatible metal selected from the group consisting of titanium, zirconia and combinations of titanium and zirconia.

8. The system of claim 1, wherein the implant fixture has a shape and form selected from the group consisting of threaded root, threadless root, blade shape, cylindrical shape, T-shape and wedge shape.

9. The system of claim 1, wherein the implant fixture comprises one or more ring-shaped extensions, and wherein each of said extensions comprises an aperture suitable for containing a fixation screw.

10. The system of claim 1, wherein the implant fixture and/or the bone block comprise one or more transverse channels, each of which is suitable in size and shape for containing a fixation screw.

11. The system of claim 1, further comprising additional means for securing the scaffold implant to the subject's bone, wherein said means are selected from the group consisting of one or more covering screws and one or more healing abutments.

12. The system of claim 1, further comprising a barrier membrane attached to a least a portion of the external surface of the scaffold implant.

13. The system of claim 12, wherein the barrier membrane is connected to the scaffold implant by elements and techniques selected from the group consisting of: folding the membrane around the scaffold implant, cement, glue, tacks, screws or pins.

14. The system of claim 12, wherein the barrier membrane is formed from a liquid which has been applied to the bone block by means of dipping, painting or spraying.

15. A method for implanting a scaffold implant system, as defined in claim 1, within the maxilla or mandible of a subject in need of such treatment, wherein said method comprises the steps of:
a) Preparing a site within, or in proximity to, the subject's alveolar bone in order to receive said implant system;
b) Inserting said implant system within the prepared site; and
c) Securing said implant system within the subject's maxillary or mandibular bone at the prepared site.

16. The method according to claim 15, wherein the step of securing the implant system within the subject's bone is achieved using one or more of the elements or techniques selected from the group consisting of: mechanical contact, fixation membrane, bone screws, tacks, biological glue and titanium pins.

17. The method according to claim 15, wherein the step of securing the implant system within the subject's bone comprises inserting and tightening a bone screw through a pre-existing hole that passes through the scaffold implant bone block and the implant fixture, and enters the subject's bone.

18. The method of claim 15, further comprising the step of attaching a dental restoration to the scaffold implant.
